(19) United States
(12) Patent Application Publication
(10) Pub. No.: US 2009/0053331 A1
(43) Pub. Date: Feb. 26, 2009

(54) Hands and feet sanitizing system

(76) Inventor: Colin W. Cook, Pepperell, MA (US)

Correspondence Address: P. Jeff Martin of The McGougan Law Firm, LLC, 1180 Hwy. 17 N., Suite 2, P.O. Box 250, Little River, SC, 29566, US

(21) Appl. No.: 11/894,442

(22) Filed: Aug. 21, 2007

(51) Int. Cl. A61K 36/61

(52) U.S. Cl. 424/725; 222/321.9

(57) ABSTRACT

A sanitizing system is provided which includes an antibacterial liquid composition formulated to sanitize and deodorize the hands, feet, and other areas of the body. The antibacterial liquid composition is adapted to be dispensed from a pump dispenser as a topical spray.

### RELATED APPLICATIONS

There are no previously filed, nor currently any co-pending applications, anywhere in the world.

### BACKGROUND OF THE INVENTION

1. Field of the Invention

The present invention relates generally to disinfectants or sanitizing agents and, more particularly, to a hands and feet sanitizing system.

2. Description of the Related Art

The U.S. Centers for Disease Control (CDC) has reported that handwashing is one of the most important means of preventing the spread of infection. It is the first line of defense for infectious diseases. Infectious diseases commonly spread through hand-to-hand contact include the common cold, flu and several gastrointestinal disorders, such as infectious diarrhea.

Inadequate hand hygiene also contributes to food-related illnesses, such as salmonella and E. Coli infection. According to the CDC, as many as 79 million Americans contract a food-borne illness each year. Of these, about 5,000 die as a result of their illness. Others experience the annoying symptoms of nausea, vomiting, and diarrhea.

Infectious diseases remain the leading cause of death and disease worldwide as well as the third leading cause of death in the United States. In addition to preventing widespread public health epidemics, regular handwashing can reduce the spread of antibiotic-resistant bacteria. The CDC estimates the cost of treating antibiotic-resistant infections in the United States is $4 billion annually.

Current methods for sanitizing the hands includes the use of bottles or containers filled with sanitizing gel to accommodate present society's increase in secondary physical interactions with other people. However, the containers as currently designed pose the challenge of dispensing the right quantity. In addition, many currently designed sanitizing gels and solutions fail to provide a bactericidal efficacy of 99.99%. Moreover, these sanitizers also fail to fully eliminate malodors from handling fish and odors caused by articles when worn on the hands and/or feet for extended periods, such as hockey gloves or boxing gloves. The current sanitizers merely “mask” the odor.

Accordingly, a need has arisen for a sanitizing system adapted for sanitizing the hands, feet, and other areas of the body having a bactericidal efficacy of 99.99% and for eliminating skin odors in a manner which is quick, easy, and efficient.

A search of the prior art did not disclose any patents that read directly on the claims of the instant invention; however, the following references were considered related.

U.S. Pat. No. 6,861,397 B2, issued in the name of Seitz, Jr. et al. discloses compositions having enhanced deposition of a topically active compound on a surface, such as skin or hair.

U.S. Pat. No. 5,744,062, issued in the name of Dahms et al. discloses balanced emulsifier blends that provide a stable oil-in-water emulsion of preselected viscosity.

U.S. Pat. No. 5,929,024, issued in the name of Stringer et al. discloses cleaning compositions which contain an ethoxylated alkylamidopropyldialkylammonium salt or an ethoxylated trialkyl ammonium salt.

U.S. Pat. No. 6,498,134 B1, issued in the name of Scheibel et al. discloses dishwashing compositions containing alkylbenzenesulfonate surfactants.

U.S. Pat. No. 6,156,712, issued in the name of Stringer et al. cleaning compositions containing cationic surfactant.

U.S. Pat. No. 6,774,099 B1, issued in the name of Scheibel et al. discloses dishwashing detergent compositions containing mixtures or crystallinity-disrupted surfactants.

U.S. Pat. No. 6,506,717 B1, issued in the name of Kott et al. discloses dishwashing compositions comprising modified alkybenzene sulfonates.

U.S. Pat. No. 6,589,516 B1, issued in the name of Eyre et al. discloses a composition suitable for use on the skin or hair comprising boswella extracts.

U.S. Pat. No. 6,379,652 B1, issued in the name of Liu et al. discloses an oral composition for reducing mouth odors comprising an orally acceptable vehicle containing a flavor system which includes a mixture of an essential oil and a coolant compound.

Consequently, a need has been felt for a composition dispensed as a spray, wherein composition is adapted for sanitizing the hands, feet, and other areas of the body having a bactericidal efficacy of 99.99% and for eliminating skin odors in a manner which is quick, easy, and efficient.

### SUMMARY OF THE INVENTION

Therefore, it is an object of the present invention to provide a sanitizing system adapted for sanitizing the hands, feet, and other areas of the body.

It is another object of the present invention to provide a sanitizing system which allows hands, feet, and other areas of the body to be sanitized and deodorized in a quick, easy, and efficient manner.

It is another object of the present invention to provide a sanitizing system adapted to eliminate malodors on the hands caused from handling fish.

It is another object of the present invention to provide a sanitizing system which includes a pump dispenser for storing and dispensing an antibacterial liquid composition.

It is another object of the present invention to provide a pump dispenser adapted to dispense the antibacterial liquid composition as a spray.

It is another object of the present invention to provide an antibacterial liquid composition having a bactericidal efficacy of 99.99% concerning Staphylococcal Aureus, Saprophyticus, Epidermidis, Methicillin Resistant Staphylococcus Aureus, and Vancomycin Resistant Enterococci.

It is another object of the present invention to provide an antibacterial liquid composition adapted to be dispensed from pump dispensers being supplied and made commercially available in a variety of volumes.

Briefly described according to one embodiment of the present invention, a sanitizing system is disclosed, wherein sanitizing system includes an antibacterial liquid composition adaptively formulated to sanitize and deodorize the hands, feet, and other areas of the body.

A pump dispenser is provided for storing and dispensing the antibacterial liquid composition as a spray. The pump dispenser comprises an elongated, circular container fabricated of a material being suitable for containing the antibacterial liquid composition.

The pump dispenser includes a cap affixed atop a head which can be pressed down with respect to a closure, to raise a ball valve and move a piston downwardly in a cylinder which is connected to the container.

A return spring is provided to resist downward pumping action by piston. The pumping of cap causes air to discharge from an outlet conduit and ball valve to rise, thereby causing antibacterial liquid composition to rise in a supply tube, past return spring up through a hollow interior channel in piston, and through the interior of head to be dispensed therefrom.

The antibacterial liquid composition is comprised of the following ingredients being suitably mixed to produce a liquid solution: a solvent; a primary antibacterial agent; a secondary antibacterial agent; an emulsion stabilizer; a humectant; a fragrance material; an essential oil, and a hydrolyzed wheat protein.

The use of the present invention allows hands, feet, and other areas of the body to be sanitized and deodorized in a manner which is quick, easy, and efficient.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a perspective view of the pump dispenser, according to the preferred embodiment of the present invention; and

FIG. 2 is a schematic sectional view of the pump dispenser shown in FIG. 1 which is adapted for dispensing the composition of the present invention as a spray.

### DESCRIPTION OF THE PREFERRED EMBODIMENT

### 1. Detailed Description of the Figures

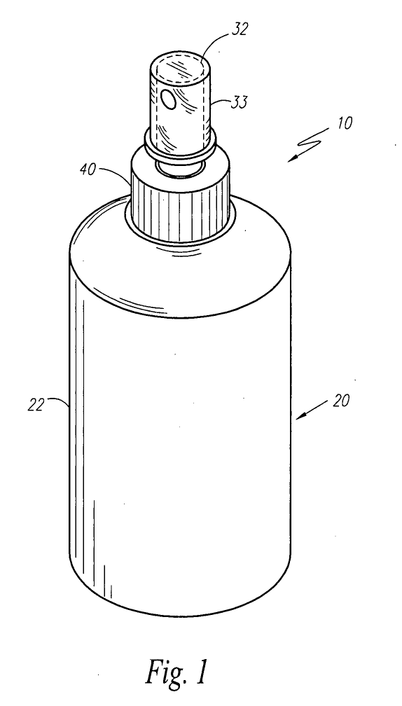

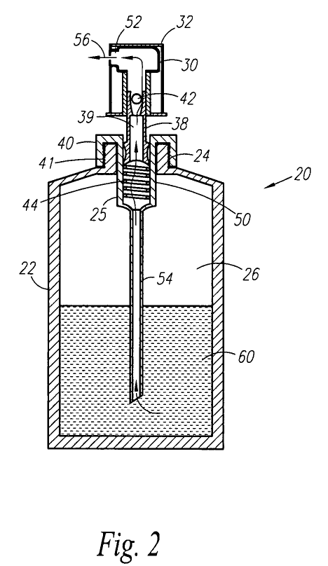

Referring now to FIGS. 1-2, a sanitizing system 10 is shown, in accordance with the present invention, comprised of a pump dispenser 20 for storing and dispensing an antibacterial liquid composition 60. The pump dispenser 20 comprises a container 22 defined of an elongated, circular configuration and is constructed of a material being suitable for containing the antibacterial liquid composition 60. It is envisioned that container 22 is constructed of a lightweight, semi-flexible material such as rubber, plastic, or plastic polymer including polyvinyl chloride (PVC). It is recognized that glass may alternatively be utilized as a construction material and be equally functional, however, in view of the significant weight differential between plastic and glass, plastic is the preferred construction material. The container 22 terminates in a threaded neck 24 having an opening 25 which leads into a hollow cavity 26.

The pump dispenser 20 includes a cap 32 affixed atop a head 30 which can be pressed down with respect to a closure 40, to raise a ball valve 42 and move a piston 38 downwardly in a cylinder 50 which is connected to the container 22. The closure 40 includes interior threads 41 adapted to threadedly mate with threaded neck 24. Tightening of closure 40 produces an air-tight sealable connection with the container 22 thereby preventing drying, leakage, spillage, or evaporation of the antibacterial liquid composition 60 stored within container 22.

A transparent cap 33 may be provided to cover cap 32. The transparent cap 33 is adapted to be removably attached to a base of cap 32 via a snap-fit connection.

A return spring 44 is provided to resist downward pumping action by piston 38. The pumping of cap 32 causes air to discharge from an outlet conduit 52 and ball valve 42 to rise, thereby causing antibacterial liquid composition 60 to rise in a supply tube 54, past return spring 44 up through a hollow interior channel 39 in piston 38, and through the interior of head 30 to be dispensed therefrom at 56.

### 2. Composition

The antibacterial liquid composition 60 is formulated to allow the user to sanitize the hands, feet, and other areas of the human body from germs, bacteria, viruses, and other harmful biological agents and microorganisms in a quick, easy, and efficient manner. The antibacterial liquid composition 60 is also formulated to eliminate malodors from handling fish and odors caused by articles when worn on the hands and/or feet for extended periods, such as hockey gloves or boxing gloves. The antibacterial liquid composition 60 is comprised of the following ingredients being suitably mixed to produce a liquid solution 62: a solvent; a primary antibacterial agent; a secondary antibacterial agent; an emulsion stabilizer; a humectant; a fragrance material; an essential oil, and a hydrolyzed wheat protein.

The composition 60 comprises more specifically the following: a solvent, wherein solvent is distilled water; a primary antibacterial agent, wherein primary antibacterial agent is isopropyl alcohol having a concentration of 70% volume/volume (v/v); a secondary antibacterial agent, wherein secondary antibacterial agent is tea tree oil (terpinen-4-ol); an emulsion stabilizer, wherein emulsion stabilizer is carbomer; a humectant, wherein humectant is glycerine having a concentration of 99.5% v/v; a fragrance material, wherein fragrance material is peppermint oil; an essential oil, wherein essential oil is marjoram; and a hydrolyzed wheat protein, wherein hydrolyzed wheat protein is skin flow.

Distilled water is present in the composition 60 at a concentration ranging from about 35 to 86% by weight, and preferably about 40-60% by weight. Isopropyl alcohol is present in the composition 60 at a concentration ranging from about 20 to 65% by weight, and preferably about 25 to 40% by weight. Tea tree oil is present in the composition 60 at a concentration ranging from about 0.5 to 2.5% by weight, and preferably about 1.0 to 2.0% by weight. Carbomer is present in the composition 60 at a concentration ranging from about 0.1 to 0.5% by weight, and preferably about 0.2 to 0.35% by weight. Glycerin is present in the composition 60 at a concentration ranging from about 1 to 40% by weight, and preferably about 5 to 20% by weight. Peppermint oil is present in the composition 60 at a concentration ranging from about 0.1 to 6% by weight, and preferably about 0.2 to 2.5% by weight Marjoram is present in the composition 60 at a concentration ranging from about 0.1 to 5% by weight, and preferably about 0.5 to 1.5% by weight. Skin flow is present in the composition 60 at a concentration ranging from about 0.01 to 2.5% by weight, and preferably about 0.05 to 2% by weight.

The antibacterial liquid composition 60 is formulated to be dispensed as a spray and applied topically on the hands, feet, and other areas of the body. Once applied, the composition 60 is rubbed into the skin within which composition 60 is absorbed and quickly dissipated.

The ingredients comprising the antibacterial liquid composition 60 are set forth in the Table below.

| INGREDIENTS | COMPOSITION BY WEIGHT |
|---|---|
| Distilled water | 35-86% |
| Isopropyl alcohol (70%) | 20-65% |
| Tea tree oil | 0.5-2.5% |
| Carbomer | 0.1-0.5% |
| Glycerin (99.5%) | 1.0-40% |
| Peppermint oil | 0.1-6% |
| Marjoram | 0.1-5% |
| Skin flow | 0.01-2.5% |

Research has shown that the antibacterial liquid composition 60 has a bactericidal efficacy of 99.99% concerning Staphylococcal Aureus (Staph A), Saprophyticus, Epidermidis, Methicillin Resistant Staphylococcus Aureus (MRSA), and Vancomycin Resistant Enterococci (VRE).

The antibacterial liquid composition 60 is adapted to be dispensed from pump dispensers 20 being supplied and made commercially available in a variety of volumes. Preferably, the antibacterial liquid composition 60 is commercially available in three-ounce pump dispensers 20.

### 3. Method of Use

A method is provided for sanitizing and deodorizing the hands, feet, and other areas of the body, wherein the method comprises the steps of: shaking vigorously the pump dispenser 20 to ensure a homogenous solution 62; applying at least two sprays of antibacterial liquid composition 60 to a desired area of the body; rubbing the antibacterial liquid composition 60 thoroughly into the skin of the desired area of the body in a manner such that an entire outer surface of the skin thereof is completely covered or enveloped with the antibacterial liquid composition 60; and allowing the antibacterial liquid composition 60 to absorb into the skin by waiting approximately one minute before contacting the skin with another object.

### 4. Operation of the Preferred Embodiment

To use the present invention, user vigorously shakes the pump dispenser 20 to ensure a homogenous solution 62. User next applies at least two sprays of antibacterial liquid composition 60 to a desired area of the body, such as the hands. User then rubs the antibacterial liquid composition 60 thoroughly into the skin of the desired area of the body in such a manner whereby an entire outer surface of the skin of the desired area of the body is completely covered with the antibacterial liquid composition 60. Finally, user allows the antibacterial liquid composition 60 to absorb into the skin by waiting approximately one minute before allowing the treated skin to come into contact with any another object.

The use of the present invention allows hands, feet, and other areas of the body to be sanitized and deodorized in a manner which is quick, easy, and efficient.

Therefore, the foregoing description is included to illustrate the operation of the preferred embodiment and is not meant to limit the scope of the invention. As one can envision, an individual skilled in the relevant art, in conjunction with the present teachings, would be capable of incorporating many minor modifications that are anticipated within this disclosure. The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be broadly limited only by the following Claims.

What is claimed is:

1. A sanitizing system comprising:
a pump dispenser, said pump dispenser comprises:
a container defined of an elongated, circular configuration, said container terminates in a threaded neck having an opening which leads into a hollow cavity;
a cap, said cap is affixed atop a head adapted to be depressed with respect to a closure in order to raise a ball valve and move a piston downwardly in a cylinder, said cylinder is connected to said container; and
an antibacterial liquid composition, said composition is stored within said container.

2. The sanitizing system of claim 1, wherein said pump dispenser is adapted for storing and dispensing said composition.

3. The sanitizing system of claim 1, wherein said closure includes interior threads adapted to threadedly mate with said threaded neck, and wherein tightening of said closure produces an air-tight sealable connection with said container, thereby preventing drying, leakage, spillage, or evaporation of said composition stored within said container.

4. The sanitizing system of claim 1, wherein said pump dispenser further comprises a transparent cap adapted to be removably attached to a base of said cap via a snap-fit connection.

5. The sanitizing system of claim 1, wherein said pump dispenser further comprises a return spring adapted to resist downward pumping action by said piston, and wherein pumping of said cap causes air to discharge from an outlet conduit and actuates said ball valve to rise, thereby causing said composition to rise in a supply tube, past a return spring up through a hollow interior channel in said piston, and through an interior of said head to be dispensed therefrom.

6. The sanitizing system of claim 1, wherein said composition is formulated to sanitize and deodorize hands, feet, and other areas of a human body from odors, germs, bacteria, viruses, and other harmful biological agents and microorganisms, said composition comprising:
(a) a solvent;
(b) a primary antibacterial agent;
(c) a secondary antibacterial agent;
(d) an emulsion stabilizer;
(e) a humectant;
(f) a fragrance material;
(g) an essential oil; and
(h) a hydrolyzed wheat protein.

7. The sanitizing system of claim 6, wherein said solvent is distilled water, said primary antibacterial agent is isopropyl alcohol, said secondary antibacterial agent is tea tree oil, said emulsion stabilizer is carbomer, said humectant is glycerine, said fragrance material is peppermint oil, said essential oil is marjoram, and said hydrolyzed wheat protein is skin flow.

8. The sanitizing system of claim 6, wherein said distilled water is present in said composition at a concentration ranging from about 35 to 86% by weight.

9. The sanitizing system of claim 6, wherein said isopropyl alcohol is present in said composition at a concentration ranging from about 20 to 65% by weight.

10. The sanitizing system of claim 6, wherein said tea tree oil is present in said composition at a concentration ranging from about 0.5 to 2.5% by weight.

11. The sanitizing system of claim 6, wherein said carbomer is present in said composition at a concentration ranging from about 0.1 to 0.5% by weight.

12. The sanitizing system of claim 6, wherein said glycerin is present in said composition at a concentration ranging from about 1 to 40% by weight.

13. The sanitizing system of claim 6, wherein said peppermint oil is present in said composition at a concentration ranging from about 0.1 to 6% by weight.

14. The sanitizing system of claim 6, wherein said marjoram is present in said composition at a concentration ranging from about 0.1 to 5% by weight.

15. The sanitizing system of claim 6, wherein said skin flow is present in said composition at a concentration ranging from about 0.01 to 2.5% by weight.

16. The sanitizing system of claim 6, wherein said composition is adapted to be dispensed as a topical spray.

17. The sanitizing system of claim 9, wherein said isopropyl alcohol has a concentration of 70% v/v.

18. The sanitizing system of claim 12, wherein said glycerin has a concentration of 99.5% v/v.

19. The sanitizing system of claim 6, wherein said composition is formulated to eliminate malodors from handling fish and odors caused by articles when worn on the hands and/or the feet for extended periods.

20. A method for sanitizing and deodorizing hands, feet, and other areas of a human body, said method comprising the steps of:
(a) shaking vigorously a pump dispenser to ensure a homogenous solution of an antibacterial liquid composition;
(b) applying at least two sprays of said antibacterial liquid composition to a desired area of the body;
(c) rubbing said antibacterial liquid composition thoroughly into a skin of the desired area of the body in a manner such that an entire outer surface of the skin thereof is completely covered or enveloped with said antibacterial liquid composition; and
(d) allowing said antibacterial liquid composition to absorb into the skin by waiting approximately one minute before contacting the skin with another object.
